(19) United States
(12) Patent Application Publication
(10) Pub. No.: US 2005/0287578 A1
(43) Pub. Date: Dec. 29, 2005

(54) Methods for RNA fluorescence in situ hybridization

(75) Inventor: Lisa Davis, Albuquerque, NM (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Exagen Diagnostics, Inc., Albuquerque, NM (US)

(21) Appl. No.: 11/159,799

(22) Filed: Jun. 23, 2005

Related U.S. Application Data
(60) Provisional application No. 60/583,568, filed on Jun. 28, 2004.

(51) Int. Cl. C12Q 1/68

(52) U.S. Cl. 435/6

(57) ABSTRACT

The present invention provides improved methods for in situ hybridization (ISH) comprising: (a) obtaining a tissue sample from a subject; (b) contacting the tissue sample with a fixative under conditions to cause fixation of the tissue sample; (c) contacting nucleic acids in the tissue sample with a detectable probe under conditions suitable to promote hybridization of the detectable probe to a target RNA in the tissue sample; (d) removing non-bound probe from the tissue sample; and (e) detecting the probe bound to the target RNA.

### CROSS REFERENCE

This application claims the benefit of U.S. Patent Application Ser. No. 60/583,568 filed Jun. 28, 2004, which is herein incorporated by reference in its entirety.

### FIELD OF THE INVENTION

The invention relates generally to nucleic acid detection.

### BACKGROUND OF THE INVENTION

While gene expression profiling (GEP) provides valuable data for identifying markers of disease states, the technology is too expensive, time consuming, technically demanding, and impractical to be used as a routine diagnostic tool. Many practitioners of the art are therefore developing methods for translating the data mining outcomes of those genome-wide gene expression datasets into diagnostic, prognostic, and/or predictive assays that measure the gene expression levels of smaller, relevant subsets of genes. Even with a focus on smaller subsets of relevant genes, one practical impediment stems from RNA's extreme vulnerability to degradation during the preparative and analytic steps of the assay. In general, current methods overcome this limitation by using tedious and expensive precautionary measures to avoid degradation when purifying and assaying RNA, such as working only in “RNA-designated facilities with RNA-designated equipment” and pre-treating solutions, glassware, plastic ware, etc. with RNAse inhibiting compounds during the purification and analytic steps. Furthermore, most gene expression data are derived from tissues, which are usually heterogeneous populations of cells, each of which contribute a unique pattern of gene expression to the tissue as whole. Hence the gene expression pattern of a tissue depends on the numbers of cells in the population expressing the gene of interest and the level of expression of each gene in the individual cell. In general, methods in the art overcome this second limitation by isolating cell types of interest from a heterogeneous population prior to assessing RNA expression. However, correct interpretation of an assay based initially on a purified cell population at the data collection and mining steps also requires that the assayed cells be purified. Such cell purification steps are frequently too cumbersome and/or expensive for routine use.

A third problem typically encountered with translation of gene expression data mining results into functional assays occurs when the assay itself is PCR-based. Many such assays are PCR-based, with the target transcripts falling into a wide abundance range, and PCR is not itself a linear assay over such wide ranges.

### SUMMARY OF THE INVENTION

The present invention provides improved methods for in situ hybridization (ISH) comprising: (a) obtaining a tissue sample from a subject; (b) contacting the tissue sample with a fixative under conditions to cause fixation of the tissue sample; (c) contacting nucleic acids in the tissue sample with a detectable probe under conditions suitable to promote hybridization of the detectable probe to a target RNA in the tissue sample; (d) removing non-bound probe from the tissue sample; and (e) detecting the probe bound to the target RNA.

### DETAILED DESCRIPTION OF THE INVENTION

The present invention provides methods for overcoming the limitations of the prior art, particularly when attempting to translate data mining results into a useful diagnostic, prognostic, or predictive assay. For example, the present invention includes a fixation step after specimen collection to eliminate the need for RNA purification. Similarly, the present invention eliminates the need for both prior cell purification steps and for intermediate PCR steps. In various embodiments, the present invention can be used to analyze gene expression levels of relevant genes in relevant cell types, and to stochiometrically measure transcript abundance at the moment of fixation.

The methods of the invention further apply to all in situ hybridization methods involving RNA detection. Thus, in one aspect, the present invention provides methods for in situ hybridization (ISH) comprising:
(a) obtaining a tissue sample from a subject;
(b) contacting the tissue sample with a fixative under conditions to cause fixation of the tissue sample;
(c) contacting nucleic acids in the tissue sample with a detectable probe under conditions suitable to promote hybridization of the detectable probe to a target RNA in the tissue sample;
(d) removing non-bound probe from the tissue sample; and
(e) detecting the probe bound to the target RNA.

As used herein, the term “subject” refers to any patient that may benefit from the diagnostic, prognostic and/or predictive tests of the invention. Preferably the subject is a mammal, and more preferably the subject is a human.

As used herein, the term “tissue sample” refers to any cellular sample taken from a subject, such as bodily fluid samples or surgical specimens taken for pathological or histological interpretation. In a preferred embodiment, the tissue sample is a bodily fluid sample, including but not limited to blood, bone marrow, saliva, sputum, throat washings, tears, urine, semen, and vaginal secretions or surgical specimen such as biopsy or tumor, or tissue removed for cytological examination. In a most preferred embodiment, the bodily fluid sample is a blood sample.

The tissue sample used in the present invention includes all the cell types present in that particular tissue sample. Thus, the methods do not involve the isolation of any sub-populations of cells in the tissue sample, as is commonly practiced in the art. In a preferred embodiment, the method further comprises identifying the cells within the tissue sample that express the gene of interest. For example, specific cells can be identified by simple dye staining methods (e.g. hematoloxylin and eosin (H&E), May Grunwald, Wright's and Giemsa stain that identify cells such as eosinophils, basophils, neutrophils, monocytes, and plasma cells present in PBMC). Furthermore, immunoflourescence or immunohistochemistry techniques can be used to detect cell surface antigens or intracellular proteins known to be specific for a given cell type, such as CD4+ or CD8+ T cells among white blood cells, or intracellular proteins, such as interleukins. Antibodies against such cell surface antigens and intracellular proteins are widely available as is known by those of skill in the art. In all of these cases, detection of the cell type is accomplished using the methods disclosed above and known to those of skill in the art. In the preferred embodiment, the cell identification will occur simultaneous with, subsequent to, or prior to, the FISH analysis. Alternatively, the cellular composition of a tissue sample can also be measured using these detection techniques in parallel assays, either on microscope slides or using alternative means, such as by flow cytometry.

Tissue samples can be used in the methods of the present invention at any volume or amount that serves the purpose of the method and is obtainable from the particular bodily fluid source. In a preferred embodiment, a bodily fluid sample used ranges between 1 uL and 10 ml; preferably between 1 uL and 3 ml; more preferably between 2 uL and 1 ml; and more preferably between 5 uL and 500 uL. It will be understood by those of skill in the art that the methods do not require the use of the entire bodily fluid sample collected from the subject.

As used herein, the term “fixative” refers to a reagent that preserves cells and tissue constituents in as close a life-like state as possible and to allows them to undergo further analytic procedures without change. Fixatives cross link the RNA molecules to other cellular molecules (proteins and other macromolecules) in their original cellular location, thus preventing diffusion, while inactivating cellular degradative RNAses. Fixation also arrests autolysis and bacterial decomposition and stabilizes the cellular and tissue constituents so that they withstand the subsequent stages of tissue processing.

The selection of an appropriate fixative is based on considerations such as the structures and entities to be demonstrated and the effects of short-term and long-term storage. Each fixative has advantages and disadvantages. Some are restrictive while others are multipurpose. In non-limiting examples, the fixative can be one or more of a buffered formalin solution, aldehydes, such as formaldehyde and, glutaraldehyde; oxidizing agents such as metallic ions and complexes, such as osmium tetroxide, chromic acid; protein-denaturing agents, such as acetic acid, methyl alcohol (methanol), and ethyl alcohol (ethanol); mercuric chloride; picric acid; or procedural such as microwaving, excluded volume fixation, and vapour fixation. Such fixatives are widely available and are known to those of skill in the art.

The target RNA can comprise one or more target RNAs, which can be any RNA for which detection is desirable. Typically the target RNA is a mRNA expression product of a gene, but could include other types of RNA sequence, such as tRNAs, rRNAs, and intracellular pathogen messenger or genomic RNA. In some cases only one species of RNA might be detected. In other cases more than one species of RNA might be detected. For example, some diagnostic assays require the identification of multiple targets to decrease false negatives and/or increase the accurate identification of positives. In other cases differential gene expression measurement of more than one gene in one or more cell might be required for interpretation. Such methods require the use of multiple detectable probes (“multiplex”). In these embodiments, it is preferred that each probe be labeled with a unique fluorochrome, allowing all the RNAs to be evaluated in one experiment.

It will also be understood by those in the art that even if only a single target RNA is being detected, it may be preferable to use multiple probes that target different regions of a single RNA target. In these embodiments, it is not necessary that the different probes be optically distinguishable, although distinguishable probes could be used.

The probe comprises a nucleic acid probe, which can comprise DNA or RNA and be single or double stranded, and which contains sequences that are complementary to the target RNA. It is preferred that such nucleic acid probes be at least 10 nucleotides in length, more preferred at least 15, more preferred at least 20, and even more preferred that the probe contains sequences complementary to the entire target RNA sequence. Thus, the probes can be synthetic polynucleotides or can be derived from genomic DNA, cDNA, etc. Such genomic DNA can be used with any accompanying repetitive sequences (preferably including competitor DNA in the hybridization), or can be modified to remove repetitive sequence elements using standard methods in the art. In a most preferred embodiment, single stranded “anti-sense” probes, which bind specifically to the RNA target, are used. In mRNA FISH (i.e. FISH to detect messenger RNA), an anti-sense probe strands hybridizes to the single stranded RNA, and in that embodiment, the “sense” strand oligonucleotide can be used as a negative control. In another embodiment, DNA probes can be used as probes, but in this embodiment, one must distinguish between hybridization to cytoplasmic RNA and hybridization to nuclear DNA. This distinction could be based on either of at least two criteria: (1) Copy number differences between the types of targets (hundreds to thousands of copies of RNA vs. two copies of nuclear DNA), which will normally create significant differences in signal intensities. In this case “control” probes targeted to either DNA only and/or to RNA only could be included in the experimental design in order to quantitate a correlation between signal intensity and target copy number. Alternatively, (2) the hybridized target RNA can be identified by the location of the fluorescent signal, based on a clear morphological distinction between the cytoplasm and the nucleus, which can be accomplished, for example, by including a nuclear DNA staining dye such as DAPI, (4,6-diamidino-2-phenylindole). RNA target hybridization will occur in the cytoplasm, which does not counter-stain with DAPI, and DNA hybridization will occur in the nucleus, which will counter-stain with DAPI. Thus, when using double stranded DNA probes, it is preferred that the method further comprises distinguishing the cytoplasm and nucleus in cells being analyzed within the tissue sample. Such distinguishing can be accomplished by any means known in the art, including other nuclear DNA staining dyes such as propidium iodide (PI) or Hoechst 33342. In this embodiment, it is preferred that the nuclear stain is distinguishable from the detectable probe. It is further preferred that the nuclear membrane be maintained, i.e. that all the Hoechst, PI, or DAPI stain be maintained in the visible structure of the nucleus.

As used herein a “detectable” probe is a probe that can be used to detect the target of interest. Such probes contain a detectable label, including but not limited to fluorescent, luminescent, or radioactive labels. In a preferred embodiment, fluorescently labeled probes are employed. Fluorophores that can be used to label a probe of interest are widely available, for example from Vysis (Abbott Laboratories, Downer's Grove Ill.), Amersham Biosciences (Piscataway N.J.), and Molecular Probes (Eugene Oreg.). Several commonly available protocols are in standard use both academically and commercially for attaching fluorescent dyes to probes.

The methods of the present invention find application in a wide variety of assays, including but not limited to diagnostic assays, prognostic assays, predictive assays, and/or therapeutic response to disease treatment, such as treatment for cancer, infectious diseases, genetic diseases, inborn errors of metabolisms, psychiatric disorders, autoimmune diseases, asthma, heart disease, high cholesterol, and high blood pressure. In a preferred embodiment, the methods are used for tumor diagnostic and/or prognostic assays.

In various non-limiting examples, (1) PBMC can also be used to detect immune response to treatment of infection; (2) Circulating blood or bone marrow can be used to identify the presence of metastatic cancer cells; (3) Circulating blood or bone marrow can be used for prognosis, and for both predicting and monitoring response to anti-tumor therapy; (4) PBMC can be used to detect organ damage as a response to auto immune disease or to infection; (5) PBMC measurement of immune response can be used to predict early response to chemotherapy. As used herein, “PBMC” refers to white cells existing in peripheral blood.

In a preferred embodiment, the tissue sample is placed on a solid support for analysis. As used herein, the “solid support” refers to any such support that can be used for the methods of the invention. In preferred embodiments, the solid support is transparent, to facilitate detection. In further preferred embodiments, the solid support is a microscope slide or a multi-well microplate. The contacting of the tissue sample with a fixative, such as a buffered formalin solution can take place either prior to, simultaneously with, or after placing the tissue sample onto a solid support. Thus, for example, a blood sample from the subject can be contacted with a buffered formalin solution prior to being placed onto the solid support, such that the RNA in the blood sample is fixed prior to the blood sample being placed on the solid support. This contacting can be done at any time after obtaining the tissue sample; preferably, the tissue sample is kept at physiological salt (to prevent cell lysis) and pH conditions (to mimic normal body pH, and to keep normal cell metabolic activity stable) until the sample is fixed. The cells could be kept at 4° C. to slow down metabolism, or could be frozen using methods that preserve cell viability, cell morphology, and other cell characteristics that are required for cell recognition, such as slow freezing in 10% DMSO. In a preferred embodiment, the contacting is done at the time of obtaining the tissue sample from the subject.

In these embodiments, the tissue samples can be stored either in the fixative or on the solid support after placing the tissue sample on the support. Alternatively, the tissue sample can be placed onto the solid support, followed by contacting with the buffered formalin solution, such that fixation of the RNA in the tissue sample occurs after placement of the sample onto the solid support. In either case, the RNA in the tissue sample is fixed and therefore stable until in situ hybridization is performed.

In various further embodiments, the tissue samples can optionally be de-proteinized (use of proteinases, for example), dehydrated, and/or rehydrated using standard methods known to those in the art.

The methods of the invention may include a nucleic acid denaturation step. For example, a denaturation step is not required when the probe is a single stranded probe, although such a denaturation step can optionally be included to reduce secondary structure of the probe and/or the nucleic acid targets. In such an optional nucleic acid denaturation step, any method for denaturing nucleic acids can be used with the methods of the invention. In one embodiment, the labeled nucleic acid probes and the nucleic acids in the tissue sample are simultaneously denatured for between 30 seconds and one hour; more preferably between 30 seconds and 30 minutes; more preferably between 30 seconds and 10 minutes; more preferably between 30 seconds and 5 minutes; even more preferably between 1 and 2 minutes. Preferred denaturation temperatures are between 90°-100° C.; more preferably between 95° and 100° C.; even more preferably between 95° and 98° C.

It is further preferred that any optional denaturation step be carried out in the solution to be used for hybridization, as discussed below. It is further preferred that the hybridization solution containing the labeled probes is applied to the tissue sample which is immobilized on the solid support. In a further preferred embodiment, coverslips, such as glass coverslips, are placed over the tissue sample-probe solution mixture, to permit uniform spreading of the probe solution (with or without a sealant between the coverslip and the solid support). In a further preferred embodiment, nucleic acids in the sample (probes plus tissue sample) are first denatured for one minute at an elevated temperature as described above, and hybridization between the probe and the target RNA occurs as the temperature decreases from the denaturation temperature to room temperature by cycling through a series of 10 degree temperature increments, holding each temperature 10 seconds, through several cycles for each pair of temperatures. For example, the slide can be cycled between 80° C. and 90° C. five times, maintaining each temperature for 10 seconds, then between 60° C. and 70° C. for ten cycles (10 seconds each), then between 50° C. and 60° C. 10×, then 30° C./40° C. 10×, and finally 25° C./30° C. 10×. Alternatively, the slides can simply be brought to 100° C.±5° C. for 1 to 2 minutes, then allowed to decrease steadily to 55° C. over a period of two to five minutes, then kept at 55° C. for 30 minutes to overnight.

In a preferred version of each of these embodiments, both the optional denaturation step and the hybridization occur in the presence of the same hybridization solution. In all of these embodiments, hybridization can be carried out in the presence or absence of competitor nucleic acid, although it is preferred that no competitor DNA be used if using either of hybridization buffers F or G under the conditions disclosed herein (see below).

The methods of the invention can be used in conjunction with any hybridization/wash buffers known in the art that are appropriate to carry out the methods. Determination of such conditions is well within the level of skill in the art. In a preferred embodiment, the methods utilize hybridization buffers disclosed in U.S. Pat. Nos. 5,750,340 and 6,022,689, incorporated by reference herein in their entirety. In a more preferred embodiment, one of hybridization buffers F or G is used, as disclosed in U.S. Pat. No. 5,750,340:

F:
10%+/−2% by weigh dextran sulfate
10%-30% (preferably 20%) by volume formamide
0.9% by weight NaCl, KCl, or other appropriate salt

G:
10%+/−2% by weigh dextran sulfate
15-25% glycerol (preferably 20%)
0.9% by weight NaCl, KCl, or other appropriate salt

The use of these hybridization buffers decreases the number of steps required for ISH. For example, other methods involve various laborious steps, separate denaturation of target nucleic acids and labeled probes, separate denaturation and hybridization procedures, and repeated dehydration of target nucleic acids with graded alcohols. Use of the preferred hybridization buffers simplifies the required steps and decreases the time required to carry out the ISH.

In a preferred embodiment, the labeled probe is dissolved in the hybridization solution. While any particular set of hybridization conditions that promote hybridization of the detectable probe to the target RNA can be used, it is preferred that simultaneous denaturation and hybridization conditions using buffers F or G, as disclosed in U.S. Pat. No. 5,750,340 and summarized below, are used.

Denaturation and Hybridization with Buffer F or G:

The labeled probe is diluted to the appropriate concentration in hybridization solution F or G and 5 to 30 uL of the probe is applied to the fixed tissue sample. It is understood that one skilled in the art will incorporate factors such as target concentration and probe characteristics when choosing the concentration and volume of the probe. The slide is then covered with a coverslip, which may or may not be sealed, depending on the hybridization time. Generally, the longer the hybridization, the more advisable is sealing the coverslip, since sealing limits evaporation of the hybridization solution.

The slides are subjected to denaturing conditions of 100° C.±5° C. for 1.5±0.5 minutes, either in a hybridization oven or on a heating plate. After denaturation, the slide can be transferred immediately to another 55° C. oven, or can be brought to 55° C. gradually by reducing the temperature of the hybridization plate or oven to 55° C. Slides can be maintained at 55° C. for 5 minutes to overnight.

In a further preferred embodiment, as disclosed in U.S. Pat. No. 6,022,689, nucleic acids in the sample (probes plus tissue sample) are first denatured for 1±0.5 min at an elevated temperature as described above, and hybridization between the probe and the target RNA occurs as the temperature decreases from the denaturation temperature to room temperature. In one embodiment, the slide is simply held at 100±5° C. for 1.5 to 2 minutes, then allowed to decrease steadily to 55° C. over a period of two to five minutes, then kept at 55° C. for 30 minutes to overnight. In another embodiment, the slide is cycled several times between pairs of 10 degree temperature increments, holding each temperature for 10 seconds. For example, after being held at 100±5° C. for 1.5 to 2 minutes, the slide is cycled 5 times between 80° C. and 90° C. (holding each temperature for 10 seconds), then between 60° C. and 70° C. ten times (10 seconds each), then 50° C. and 60° C. 10×, then 30° C./40° C. 10×, and finally 25° C./30° C. 10×. The cycling process occurs over 30 to 60 minutes, and the slide can then be immediately washed (below), or kept at 30° C. for up to 18 hours or returned to 55° C. for 30 minutes to overnight, until the washing step (below).

Post Hybridization Wash:

Similarly, any wash conditions can be used that minimize the retention of unbound probe to the tissue sample on the solid support. As will be appreciated by those of skill in the art, this step does not require that all unbound probe is removed, but simply that enough unbound probe is removed to permit adequate detection of the bound probe to the target RNA.

In a preferred embodiment, the coverslips are removed and the solid support is washed with 50% formamide in 0.45% NaCl for 3 minutes at 38° C., and then for 5 minutes in 0.9% NaCl at 38° C. Alternatively, the hybridized slides are washed in formamide-free 0.1-0.2% NaCl at 60° C. for 5 minutes and then for another 3 minutes in fresh 0.1-0.2% NaCl at 60° C. The solid supports are then preferably air dried prior to detection.

Any method of detecting the label on the detectable probes can be used to detect the probe bound to the RNA target. In a preferred embodiment, the detection comprises visualization of the probe in the cell by fluorescent microscopy. In this embodiment, it is preferred that after the wash step is completed, the cells are stained in order to visualize individual fluorescent signals in individual cells. Such staining can include nuclear staining or other staining, such as with a fluorescent cell surface marker. In one example, the cells in the bodily fluid sample are counter-stained with Hoechst 33342, 4,6-diamidino-2-phenylindole (DAPI) or propidium iodide (PI) solution.

In a further embodiment, the fluorescent signal detection is further accompanied by identification of the cell in which the signal is detected. Cell identification can be accomplished simultaneously with the RNA fluorescent signal detection by, for example, dual hybridization with a fluorescent cell-specific surface antigen. Simultaneous cell identification can also be accomplished by co-hybridizing the test probe with a second fluorescent probe for a cell-specific gene known to be expressed in the identified cell.

In yet another embodiment, the detection further comprises qualitative or quantitative analysis of the target RNA present in individual cells. For example, the location of the target RNA in cytoplasmic RNA of the clinical specimen is visualized using fluorescence microscopy (RNA-FISH). Fluorescence signals can be visualized with a fluorescence microscope equipped with a triple band-pass filter and a 20× or 40× dry objective lens. Signals can also be viewed at 100× under oil.

Further, the target RNA can be quantified using commercially available hardware and software for fluorescent signal detection and quantification, image capture, processing, and storage. Such hardware and software are available from, for example, Applied Imaging (San Jose Calif.) or MetaSystems (Altlussheim Germany). These imaging systems have been designed to quantify individual signals, and they can accommodate the signal overlap that sometimes occurs in dual FISH hybridization. These commercially available systems can also quantify the diffuse signals that occur in cytoplasmic RNA hybridization as well as the discrete signals that occur in chromosomal DNA hybridization.

### EXAMPLE 1

### Blood Cell Fixation

In a non-limiting example of the methods of the invention comprising blood cell fixation, three issues for consideration are: Blood must not coagulate before slides are made; (2) Cells must be fixed; and (3) Cells must adhere to the solid support. Thus, in one example, blood is collected in EDTA or heparin tubes. The blood is fixed immediately or up to 24 hours later. There are two basic methods for fixing/attaching cells to slides:

(1) Cells are fixed after the blood smears are made:

Blood smears are prepared from EDTA tubes directly on poly-L-lysine coated microscope slides. The slides are pre-coated with lysine or another adherent (including but not limited to collagen, laminin, CAPS, amino-silane; such slides are available from Fisher, VWR, and Corning) After the blood is smeared on the slide, the slides are air-dried for 2-5 minutes until dry, and then placed into coplin jars filled with 10% formalin. In this case, the cells are not fixed until they are placed in the jars containing formalin

(2) Cells are fixed before attaching to the slides:

Cells can be fixed by mixing small aliquots of the blood sample with an approximately equal volume of formalin solution (i.e. up to a 70:30 ratio of either blood:formalin, or formalin:blood, preferably no more than a 60:40 ratio; more preferably approximately a 50:50 ratio) and left at room temperature for 5 minutes to overnight. The blood is then smeared onto standard untreated microscope slides. After drying, the slides are placed in coplin jars containing 10% formalin. Poly-L-lysine slides can also be used with cells that are fixed prior to attachment.

If cells are to be applied to untreated slides, the cells must be fixed first; if cells are applied to treated slides, the cells can be fixed before or after application to the slide. Slides can be stored in coplin jar in formalin for one or two weeks at room temperature, or transferred immediately to 4° C. or −80° C., where they can be stored indefinitely.

### EXAMPLE 2

### Hybridization with a Single, Direct Label Probe

This method illustrates identification of cells in a blood smear that express the IgG heavy chain gene, and quantification of expression of the IgG gene in the cell. A sample of blood is collected in an EDTA anticoagulant tube, and the tube is inverted several times. A drop of blood is applied to a poly-Lysine coated slide and the blood is smeared across the microscope slide by dragging the edge of another microscope slide through the drop of blood and across the slide. After smearing the blood, the cells do not touch each other and the smear is “feathered” at the end of the dragging motion. After air drying, the slide is placed in a coplin jar or other staining dish containing 10% formalin in buffered saline. After 30 minutes the slide is removed from the formalin and air dried.

A sense strand oligonucleotide probe for the IgG heavy chain mRNA, direct-labeled with FITC (GeneDetect, Sarasota Fla.) is suspended in hybridization solution G at a concentration of 200 ng/ml, and 10 uL is applied directly to the blood smear, and covered with a coverslip.

The slide is heated to 98° C. for 1.5 minutes and then transferred to a 55° C. oven for 60 minutes. Alternatively, the denaturation and hybridization steps can be accomplished on a single instrument that has a programmable temperature controlled surface (such as a Hybrite, Vysis, Inc.). In this case the surface is programmed to hold a temperature of 95° C. for 1.5 minutes, and then programmed to decrease to 55° C. and hold for 60 minutes. (Other commercially available thermocyclers that are designed for “in situ PCR” are also suitable for this application).

After hybridization, the slide is washed of excessive probe in 0.1-0.2% NaCl at 60° C. for 5 minutes and then for another 3 minutes in fresh 0.1-0.2% NaCl at 60° C. The slide is then counterstained with 20 ng/ml DAPI in antifade. Hybridization signals are viewed through a triple band pass filter on an Olympus BH-2 fluorescent microscope using a 40× objective. Cells containing diffuse green FITC fluorescence throughout their cytoplasm are considered positive for IgG. The cytoplasm is distinguished from the blue DAPI stained nuclei. Positive cells in a defined field of view are counted and the number of positive cells in the sample is calculated according to the area of the field of view. Cells expressing the IgG gene are reported as a percentage of the DAPI stained cells. Quantification of the RNA levels in individual cells is determined by viewing, imaging, capturing, and quantifying the fluorescent signal with MetaSystems' Metafer-Metacyte slide scanning software and signal quantification software (MetaSytems, Altlussheim, Germany) modified with a classifier for quantitating cytoplasmic mRNA.

### EXAMPLE 3

### Multiplex Hybridization: Dual Indirect-Labeled Probes

The following example illustrates simultaneous identification and quantification of cells co-expressing the IgG heavy chain and IL-12 genes in a blood smear, and quantification of the gene expression levels of each gene in the expressing cells. A sample of blood is collected from the patient in an EDTA anticoagulant tube, smeared on a poly-Lysine coated slide, and fixed in formalin as described above in Example 1. After a 30 min fixation, the slide is air dried.

The biotin-labeled IgG probe and a digoxin-labeled IL-12 probe (both sense strand oligonucleotide probes) (GeneDetect, Sarasota Fla.), are suspended together in hybridization solution G at concentrations of 200 ng/ml each, and 10 uL of the mixture is applied directly to the blood smear, overlaid with a coverslip, and the coverslip is sealed with contact cement. The slide is heated to 98° C. for 1.5 minutes and then transferred to a 55° C. oven overnight.

After hybridization, the slide is washed of excessive probe in 0.1-0.2% NaCl at 60° C. for 5 minutes and then for another 3 minutes in fresh 0.1-0.2% NaCl at 60° C. The biotin labeled IgG probe is then detected using an avidin-anti-avidin-FITC sandwich detection method and the digoxin labeled IL-12 probe is detected using anti-digoxin conjugated to rhodamine (Roche), both according to manufacturers' suggestions. After detection, the slide is counterstained with DAPI (20 ng/ml in antifade).

Hybridization signals are viewed initially through a triple band pass filter on an Olympus BH-2 fluorescent microscope using a 40× objective. Cells expressing only IgG are identified by their cytoplasmic FITC (green) fluorescence, and cells expressing only IL-12 are identified by their cytoplasmic rhodamine (red) fluorescence. Individual cells expressing only one gene are quantified as a percentage of the DAPI stained cells. Cells expressing both IgG and IL-12 are viewed as a mixture of red and green cytoplasmic fluorescence (yellow) and the dual expressing cells are expressed as a percentage of DAPI stained cells. IgG and IL-12 gene expression levels in each cell type are quantified by viewing, imaging, capturing, and quantifying the fluorescent signal with MetaSystems' Metafer-Metacyte slide scanning software and signal quantification software (MetaSytems, Altlussheim, Germany) modified for with a classifier for quantitating cytoplasmic mRNA. The classifier discriminates the green and red fluorescence and reports each signal independently of the other.

### EXAMPLE 4

### Multiplex, Direct-Label, Dual Hybridization: Simultaneous Measurement of Gene Expression Level and Cell Identification

In this example, the first of the two probes hybridizes to the gene for IL-6 and is used to quantitate its gene expression level, and the second of the two probes, for the IgG heavy chain, identifies the cell as a B-cell. The IgG heavy chain is direct-labeled with FITC and the IL-6 probe is direct labeled with rhodamine (GeneDetect, Sarasota Fla.). As in examples 1 and 2 above, a blood smear is made on poly-L-Lysine slides. Each oligonucleotide anti-sense probe is diluted into hybridization buffer G at a concentration of 200 ng/ml, and 10 uL of the mixture is added to the air-dried blood smear. A coverslip is laid on top of the hybridization solution and sealed. The slide is heated to 98° C. for 1.5 minutes and then transferred to a 55° C. oven overnight.

After hybridization, the slide is washed of excessive probe in 0.1-0.2% NaCl at 60° C. for 5 minutes and then for another 3 minutes in fresh 0.1-0.2% NaCl at 60° C., then counterstained with DAPI. Hybridization signals are viewed initially through a triple band pass filter on an Olympus BH-2 fluorescent microscope using a 40× objective. Cells expressing only the IL-6 gene are identified by their cytoplasmic rhodamine (red) fluorescence, and cells expressing only the IgG heavy chain (B-cells) will be identified by their cytoplasmic FITC (green) fluorescence. B-cells that are also expressing the IL-6 gene will be identified by the combination of red and green fluorescence (which will appear yellow under the triple band pass filter). Individual cells expressing both genes are quantified as a percentage of the DAPI stained cells. IgG and IL-6 gene expression levels in individual dual expressing cells are quantified by viewing, imaging, capturing, and quantifying the fluorescent signal with MetaSystems' Metafer-Metacyte slide scanning software and signal quantification software (MetaSytems, Altlussheim, Germany) modified for with a classifier for quantitating cytoplasmic mRNA. The classifier discriminates the green and red fluorescence and reports each signal independently of the other.

1. A method for in situ hybridization (ISH) comprising:
(a) obtaining a tissue sample from a subject;
(b) contacting the tissue sample with a fixative under conditions to cause fixation of the tissue sample;
(c) contacting nucleic acids in the tissue sample with a detectable probe under conditions suitable to promote hybridization of the detectable probe to a target RNA in the tissue sample;
(d) removing non-bound probe from the tissue sample; and
(e) detecting the probe bound to the target RNA.

2. The method of claim 1, wherein the conditions suitable to promote hybridization comprise denaturing the nucleic acids in the tissue sample.

3. The method of claim 1 further comprising identifying specific cell types within the tissue sample, wherein step (e) comprises detecting the probe bound to the target RNA in one or more cell types in the tissue sample.

4. The method of claim 1, wherein the tissue sample is placed on a solid support.

5. The method of claim 4, wherein the solid support is selected from the group consisting of a microscope slide and a multi-well microplate.

6. The method of claim 4 wherein the fixation is carried out prior to placing the tissue sample on the solid support.

7. The method of claim 4 wherein the fixation is carried out after placing the tissue sample on the solid support.

8. The method of claim 1 wherein the tissue sample comprises a bodily fluid.

9. The method of claim 8 wherein the bodily fluid comprises a blood sample.

10. The method of claim 1, wherein the method is used in a diagnostic, of prognostic, or predictive assay.

11. The method of claim 1, wherein the method is used to predict or monitor therapeutic response to disease treatment.
